(19) United States
(12) Patent Application Publication
(10) Pub. No.: US 2025/0361460 A1
(43) Pub. Date: Nov. 27, 2025

(54) A LIQUID-LIQUID-SOLID EXTRACTION PROCESS FOR ISOLATING NATURAL PRODUCTS FROM A FEEDSTOCK STREAM

(71) Applicant: Neste Oyj, Espoo (FI)

(72) Inventors: Jeffrey Scott KANEL, Porvoo (FI); David Robert BRYANT, Porvoo (FI); Cecilia Alexandra STILL, Turku (FI)

(73) Assignee: Neste Oyj, Espoo (FI)

(21) Appl. No.: 18/874,440

(22) PCT Filed: Jun. 12, 2023
(86) PCT No.: PCT/US2023/025033

§ 371 Date: Dec. 12, 2024

Related U.S. Application Data
(60) Provisional application No. 63/351,651, filed on Jun. 13, 2022.

(51) Int. Cl. C11B 1/10; B01D 11/04; B01D 17/02

(52) U.S. Cl. CPC C11B 1/102; B01D 11/043; B01D 11/0434; B01D 11/0492; B01D 17/02

(57) ABSTRACT

A liquid-liquid-solid extraction process is disclosed for isolating natural products from a feedstock stream containing a biomass in an aqueous salt solution. The process includes forming a dispersion by contacting the feedstock stream with an extraction solvent in an extraction zone; passing the dispersion to a separation zone; separating the dispersion into multiple layers at a temperature of about 90° C. or less, the layers including: a solvent extract layer, a raffinate layer and a rag layer, and isolating at least part of the solvent extract layer, at least part of the raffinate layer and/or at least part of the rag layer.

### FIELD

The present disclosure relates to a liquid-liquid-solid extraction process for isolating natural products from a feedstock stream containing a biomass in an aqueous salt solution.

### BACKGROUND INFORMATION

Extraction of valuable components, such as oils and carotenoids, from biomass is known to be accomplished by drying the biomass and then subjecting the dried biomass to a leaching process (which is commonly called dry extraction). With this method, dried biomass is intimately contacted with a hydrocarbon solvent, such as hexane, to extract the oils and carotenoids. The spent biomass, depleted in oil and carotenoids, is separated from the extract by a solid-liquid separation process such as filtration or pressing. When algal biomass is extracted using this leaching process, the algal biomass is first dried and often pelletized before the leaching process can be utilized. Significant energy is involved in drying the algal biomass prior to extraction because a mechanical separation process can only concentrate algal biomass to about 10-20 wt % biomass in a wet paste. Having to remove 80 to 90% of the weight of the wet paste by drying is an overwhelming negative from a greenhouse-gas generation perspective. Thus, there is a need at an industrial scale to perform a robust extraction without evaporating so much water.

In addition, if a marine species is being extracted, the growth medium will contain salt, which is not separated from the biomass in a drying step. Thus, this salt is sent along with the biomass to the dry extraction process. Most leaching machines are constructed of stainless steel and include many moving parts which are attacked via corrosion in the presence of salts. Furthermore, if the salt to algal biomass ratio is elevated to greater than 1:10, then a substantial amount of the leaching equipment's volume is consumed by salt-which lowers the leaching machine's capacity. This is a further unwanted consequence of having salt present in the feedstock stream. A robust extraction process that can start with wet algal biomass instead of dried algal biomass would address these issues.

There is an increasing interest in using algal biomass as a key intermediate for a plethora of sustainable products, such as a source of renewable energy, as a mode to safely and efficiently capture carbon dioxide from the atmosphere for carbon sequestration, as a source of natural carotenoids and as a renewable source of chemical intermediates. For example, an algal concentrate that is produced by a harvester is often passed through an extraction process to separate the algal oil from the algal biomass. The algal oil can be a source of valuable products including carotenoids, fatty acids, and other lipids. The algal biomass can also be a source of valuable products, including protein, animal feeds, soil builder, feed for fermentation, and fuel. A more robust extraction for the separation of oil and carotenoids from algae would allow products to be obtained with high purity and high yields. The more robust extraction also maximizes the economic return from the venture.

U.S. Pat. No. 4,341,038 discloses a method to obtain oil products from algae by growing microalgae, harvesting the algae, extracting the oil from the algae, and recovering the oil and algal residue. An extraction step takes place at a temperature between 28° and 350° C. Extraction at this temperature degrades valuable carotenoids found in the algal oil. This patent does not disclose separation of the solvent from the algal biomass and an effect that temperature can have on that separation.

U.S. Pat. No. 4,680,314 discloses a process to produce a carotene dissolved in edible oil by concentrating algae, adding oil to the algae, forming an emulsion at a temperature sufficient to extract carotene from the algae, and separating the oil phase containing carotene from the water phase containing algae. In this process, centrifugation is disclosed to separate the phases created in the extraction process. However, centrifugation equipment is expensive, involves a high level of maintenance and is energy intensive.

U.S. Pat. No. 5,378,369 discloses a solvent-extraction of a beta carotene from algae into a vegetable oil by mixing the oil and aqueous algal suspension, allowing the beta carotene to dissolve into the oil, and separating the oil and aqueous phases by passing the oil phase through a semipermeable membrane. Using a semipermeable membrane to separate the phases can be feasible at a small scale. However, scaling up membrane separation processes for applications such as industrial production of algal oil for biofuels or production of large quantities of carotenoids for dietary supplements, is difficult.

U.S. Pat. No. 5,951,875 discloses a process for dewatering and extracting carotenoids from an aqueous suspension of microalgae by rupturing the cells, concentrating the cells in an adsorptive bubble separation process, contacting algal concentrate with a solvent, phase separating the extract, algal residue, and raffinate, and concentrating the carotenoids. Gravity settling is disclosed as a separation technique to separate the phases. While gravity settling can cost less than centrifugation, it involves a large amount of time to achieve adequate separation of the phases at ambient temperature. This patent does not disclose a temperature at which the extraction and separation processes occur.

To address the foregoing issues, a more economical and efficient process for isolating natural products from a biomass is desirable whereby, for example, an industrial scale, robust extraction can be achieved.

### SUMMARY

Disclosed herein is a liquid-liquid-solid extraction process for the isolation of natural products from a feedstock stream containing a biomass in an aqueous salt solution, the process including (i.e., comprising): forming a dispersion by contacting (e.g. by intimately contacting) the feedstock stream with an extraction solvent in an extraction zone; passing the dispersion to a separation zone; separating the dispersion into multiple layers at a temperature of about 90° C. or less, the layers including: a solvent extract layer containing at least one hydrophobic natural product and the extraction solvent, a raffinate layer containing the aqueous salt solution, and a rag layer containing a lipid-depleted biomass; and isolating at least part of the solvent extract layer, at least part of the raffinate layer and/or at least part of the rag layer.

### BRIEF DESCRIPTION OF THE DRAWINGS

FIG. 1 shows a flow diagram of an exemplary embodiment of a liquid-liquid-solid extraction process for the isolation of natural products from a feedstock stream in a manner as disclosed herein. The dashed lines represent optional steps which can occur in the depicted exemplary embodiment.

FIGS. 2A and 2B show exemplary counter-current column extraction embodiments. The dashed lines represent optional steps which can occur in the depicted exemplary embodiments.

FIG. 3 depicts a graph demonstrating the effect temperature has on the decantation time of the solvent extract layer, the rag layer and the raffinate layer. The stars represent the points where the coalescence or decantation is essentially completed, and there is essentially no further change in the elevation of the two interfaces.

### DETAILED DESCRIPTION

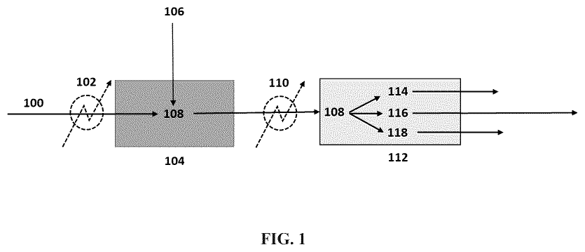

FIG. 1 shows a liquid-liquid-solid extraction process for the isolation of natural products from a feedstock stream containing a biomass in an aqueous salt solution. In this embodiment, a feedstock stream (100) can undergo a heat exchange (102) before entering an extraction zone (104).

Once in the extraction zone (104), the feedstock stream (100) contacts an extraction solvent (106). The contact between the feedstock stream (100) and the extraction solvent (106) forms a liquid-liquid-solid dispersion (108).

The dispersion (108) can pass through a heat exchange step (110) before entering a separation zone (112).

In the separation zone (112), the dispersion (108) separates into multiple layers at a temperature within a temperature range of less than or about 90° C., the layers including: a solvent extract layer (114) containing at least one hydrophobic natural product and the extraction solvent, a raffinate layer (118) containing the aqueous salt solution, and a rag layer (116) containing a lipid-depleted biomass. In exemplary embodiments, with the rag layer (116) is formed between the solvent extract layer (114) and the raffinate layer (118).

After the layers have separated, at least part of the solvent extract layer (114), at least part of the raffinate layer (118) and/or at least part of the rag layer (116) is isolated for further processing.

It has been surprisingly discovered that biomass wet with an aqueous salt solution can be intimately contacted with a solvent system to generate a three-phase system including a raffinate layer (containing an aqueous phase that is depleted in biomass), a solvent extract layer (containing one or more hydrophobic natural products derived from the biomass, such as carotenoids), and a rag layer that contains a lipid-depleted biomass.

The separation of the three-layer systems has been difficult to accomplish, and this has forced companies to use dry extraction processes. In dry extraction processes, the biomass is separated from the aqueous solution by drying prior to charging it to the extraction zone. The drying step is expensive and requires significant energy input. This disclosure not only has overcome the challenge of efficiently extracting natural products from a biomass, but it also provides a liquid-liquid-solid extraction process which can be efficiently performed under a gravitational field. This can significantly reduce the capital and energy costs of performing this extraction process. It has also been surprisingly discovered that decantation of a dispersion containing an aqueous salt solution possessing a biomass and an extraction solvent can be performed in multiple steps of decanters or extraction columns, or combinations thereof.

It is further surprising that a majority of hydrophobic natural products can be removed from the biomass and transferred into the solvent extract layer in an amount of time that renders this step to be economically attractive. If the concentration of biomass in an extractor feed is properly selected, the extraction equipment used can be any that are commonly used in liquid-liquid or liquid-liquid-solid extraction processes.

Such equipment include, but are not limited to, mixer-settlers, counter-current extraction columns, centrifugal extractors, membrane extractors, and extractors that rely upon non-standard contact methods including electrical fields, ultrasonic waves, microwave waves, and combinations thereof. The extraction can be performed at conditions including temperatures between ambient and 100° C., pressures from ambient to supercritical conditions of the extraction solvent, pH values as desired (e.g. between 4 and 11, between 4 and 10, between 5 and 10, or between 6 and 10), and/or variations and combinations that do not degrade the protein, oils, and carotenoids.

It has also been discovered that a solvent extract layer can be efficiently washed to remove any residual salt in a liquid-liquid-solid extraction process when the extract layer is intimately contacted with an aqueous solution.

Accordingly, the present disclosure relates to a liquid-liquid-solid extraction process for isolating natural products from a feedstock stream containing a biomass in an aqueous salt solution.

The expression “liquid-liquid-solid extraction” refers to a method wherein a liquid dispersion containing a biomass that includes one or more natural products is (e.g. intimately) contacted with an extraction solvent capable of extracting one or more of the hydrophobic natural products from the biomass.

The expression “feedstock stream” refers to a stream originating from a feedstock source containing a biomass solution or suspension that includes natural products.

In exemplary embodiments, the feedstock stream is a stream originating from at least one or a combination of plant, algae, micro-organism, bacteria, or microalgae feedstock sources. Suitable algae or microalgae feedstock sources can be derived from reactors that include, but are not limited to, tubular reactors, photobioreactors, enclosed raceways, covered ponds, open raceways, open ponds, earthen ponds, ponds in greenhouses, clear plastic bags hung either indoors or outdoors, fermenters, naturally occurring bodies of water, solar salt ponds, and combinations thereof.

In exemplary embodiments, the feedstock stream can contain a biomass concentration from about 0.5 wt % to about 10 wt %, from about 0.1 wt % to about 20 wt % and/or from about 1 wt % to about 7 wt %.

The feedstock stream can undergo conditioning processes before advancing to the liquid-liquid-solid extraction process. These conditioning processes can include, but are not limited to, fracking, adsorptive bubble separation, filtration, deep bed filtration, belt pressing, screw pressing, centrifugation, adsorption, sedimentation, mechanical floatation, froth flotation, flocculation and combinations thereof. Examples of these and other conditioning processes and equipment which can be used to condition the feedstock stream before the extraction process can be found in U.S. Pat. Nos. 5,541,056; 4,554,390; 4,115,949; 5,951,875; 4,680,314; 6,524,486; 6,405,948; 5,776,349; 6,000,551; 8,512,998; 4,397,741; 4,938,865; 5,188,726; 5,332,100; WO 2008/156,795; WO 2008/156,835; U.S. Pat. Nos. 4,981,582; 5,167,798; all the contents of which are incorporated herein by reference in their entireties.

In exemplary embodiments, prior to an adsorptive bubble separation process, the feedstock stream can be flotation conditioned for a number of reasons. Suitable flotation conditioning processes that can be used prior to the adsorptive bubble separation unit include, but are not limited to, adding a flotation aid, adding a frother, adding a collector, adding an activator, and combinations thereof.

Collectors selectively render one or more of the species of particles in the feed hydrophobic, thereby assisting in the process of collection by gas bubbles. Activators aid the adsorption of the collector to certain particles increasing the number of those particles which become hydrophobic. Depressors inhibit the adsorption of the collector to certain undesired particles decreasing the number of those particles which become hydrophobic. Also, frothing agents and frothers may be added to the feedstock stream to assist in the formation of a stable froth on the surface of a liquid.

Sedimentation process can include the addition of alum and/or lack agitation of the feedstock stream. The addition of ferric chloride can also be include in sedimentation processes to cause flocculation. Any polymer or ions that cause flocculation can also be used during sedimentation processes. Cyclones can also be used to accelerate the rate of sedimentation. Any sedimentation equipment known in the art can be used to separate the flocculated natural products from the aqueous salt solution prior to an adsorptive bubble separation process.

Adsorption can be used as a conditioning process to reduce the volumetric flow of the feedstock stream to an adsorptive bubble separation unit. Some feedstock, for example Dunaliella salina, can be concentrated by adsorbing the algae onto a hydrophobic surface, and then desorbing the algae with another fluid. Thus, adsorption can be used to preconcentrate the feedstock stream.

Deep bed filtration can be used to preconcentrate the feedstock stream prior to an adsorptive bubble separation process. Deep bed filtration relies upon a bed of granular media, usually sand, through which the feedstock stream containing natural products flows downward under gravity. The natural products are deposited in the pores of the granular media and in the interstitial spaces between the grains of media. Deep bed filtration should not be confused with straining filtration. Straining takes place on the surface of a mesh or fabric, and is only suitable to preconcentrate a feedstock stream with natural products that will not blind the filtration equipment.

Adsorptive bubble processes can include a step of rendering material or natural products within the feedstock stream hydrophobic by treating particle surfaces with chemicals, or other techniques that selectively modify the material or natural products to be separated. In some cases, the particles or natural products are not initially hydrophobic, and need to be rendered hydrophobic to be separated or harvested from the feedstock stream.

A flocculating agent can be utilized during adsorptive bubble separation processes to cause accumulations of feedstock or natural products to float out during adsorptive bubble processes.

The feedstock stream or the biomass included in the feedstock stream can also be subjected to a cell rupturing process before proceeding to the liquid-liquid-solid extraction processes. The feedstock can include cellular material which contains natural products. In these instances, rupturing the cell wall and/or cell membrane of the cellular material can release natural products that can be purified in later processes. Cell rupturing can be achieved by a number of methods which include, but are not limited to, chemical, physical or mechanical methods. Chemical methods can include enzymatic digestion, detergent solubilization, lipid dissolution with a solvent, and alkali treatment (lipid saponification). Physical methods can include osmotic shock, decompression, sonication, heat treatment, and freeze-thawing. Mechanical methods can include grinding, high shear homogenization, passing the feedstock stream across a pressure drop, and pressure extrusion.

Other cell disruption process which can be used include pumping the feedstock stream at high pressures through a restricted orifice valve. An equipment which can perform this disruption method is, as an example, the MICROFLUIDIZER™ cell disruption equipment of Microfluidics, Newton, MA, US, which utilizes pressures of about 5,000 to 40,000 psig (345-2760 bar).

A mill, such as a vibratory mill, can also be used to rupture cellular material in the feedstock stream.

In exemplary embodiments wherein the feedstock stream contains algae or microalgae, fracking processes can be performed on the feedstock stream before the liquid-liquid-solid extraction process. The partial rupturing of algae is referred to as fracking. Fracked algae are preferable to completely ruptured algae due to the difference in size of the resulting particles. Particles resulting from fracking algae are larger than the particles resulting from the complete rupturing of algae and thus adsorptive bubble separation processes could be more effective when larger particles are present. Fracking the algae or microalgae can produce fracked cells possessing hydrophobic components while still retaining a significant portion of the intracellular material within the cellular membrane. This can result in increased recovery of the intracellular material. Fracking can take place in any device known in the art in which algae or microalgae can be partially ruptured including, but not limited to, a vibratory mill, a French press, a pump, an agitated vessel, or combinations thereof.

The algae or microalgae which can be present in the feedstock stream can be algae from the divisions of Chlorophycophyta, Phaeophycophyta, Chrysophycophyta, Cyanophycophyta, Cryptophycophyta, Pyrrhophycophyta and/or Rhodophycophyta, which are adaptable to saline water as a growth medium; or microalgae species selected from, but not limited to, Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria sp., Fragilaria sublinearis, Gracilaria sp., Haematococcus pluvialis, Hantzschia sp., Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea Nostoc commune, Nostoc flagellaforme, Pavlova gyrens, Peridinium sp., Phaeodactylum tricornutum, Pleurochrysis carterae, Porphyra sp., Porphyridium aerugineum, Porphyridium cruentum, Prymnesium sp., Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema sp., Skeletonema costatum, Spirogyra sp., Schiochytrium limacinum, Stichococcus bacillaris, Synechoccus sp., Tetraselmis sp., Tolypothrix sp., and genetically-engineered varieties or combinations (mixtures, or mixed cultures) of these microalgal species. In exemplary embodiments, the algae or microalgae is selected from the group including Dunaliella sp., Dunaliella bardawil, Dunaliella salina, Dunaliella tertiolecta, Dunaliella parva and Dunaliella viridis, and any combination thereof. In exemplary embodiments, the algae or microalgae is Dunaliella salina.

The algae or microalgae which can be present in the feedstock stream can further include any microalgal species (including diatoms, coccolithophorids and dinoflagellates) selected from, but not limited to, Amphora sp., Ankistrodesmus sp., Arthrospira (Spirulina) plantesis, Botryococcus braunii, Chlamydomonas sp., Chlamydomonas reinhardtii, Chlorella protothecoides, Chlorella sp., Closterium sp., Cosmarium sp., Crypthecoddinium cohnii, Cyclotella sp., Dunaliella salina, Dunaliella tertiolecta, Haematococcus pluvialis, Hantzschia sp., Nannochloris sp., Nannochloropsis sp., Navicula sp., Neochloris oleoabundans, Nitzschia sp., Phaeodactylum tricornutum, Scenedesmus sp., Schiochytrium limacinum, Stichococcus sp., Tetraselmis suecica, and Thalassiosira pseudonana, and genetically-engineered varieties or combinations (mixtures, or mixed cultures) of these microalgal species.

The algae or microalgae which can be present in the feedstock stream can also include algae with flagella, cilia and/or eyespots. Flagella are a tail-like projection that protrudes from the cell body of certain algae and functionsmin locomotion. Cilia are an adaptation that allows independent cellular creatures, like algae, to move around in search of food. Photosensitive eyespots are found in some free-swimming unicellular algae. Photosensitive eyespots are sensitive to light. They enable the algae to move in relation to a light source. Such algae have the capability of independent motion, phototaxis, and can move towards the surface during daylight. Phototaxis is the movement of microalgae in response to light. For example, certain algae (e.g., Dunaliella) can perceive light by means of a sensitive eyespot and move to regions of higher light concentration to enhance photosynthesis.

The algae or microalgae which can be present in the feedstock stream also include marine algae that thrive at salt concentrations above that found in seawater. Suitable marine algae can be selected from, but are not limited to, Amphora sp. (diatom), Arthrospira sp., Arthrospira (Spirulina) obliquus, Arthrospira (Spirulina) platensis, Chlorella sp., Chlorella fusca, Chlorella protothecoides, Chlorella pyrenoidosa, Chlorella stigmataphora, Chlorella vulgaris, Chlorella zofingiensis, Dunaliella sp., Dunaliella bardawil, Dunaliella salina, Dunaliella tertiolecta, Dunaliella viridis, Isochrysis galbana, Microcystis sp., Nannochloropsis sp., Nannochloropsis salina, Navicula sp. (diatom), Navicula saprophila (diatom), Nitzschia laevis (diatom), Nitzschia alba (diatom), Nitzschia communis (diatom), Nitzschia paleacea (diatom), Nitzschia closterium (diatom), Nitzschia palea, (diatom), and genetically-engineered varieties or combinations (mixtures, or mixed cultures) of these algal species.

In exemplary embodiments, the algae is microalgae. In other exemplary embodiments, the algae or microalgae have not been genetically modified or do not originate from genetically engineered algae or microalgae.

All of the possible algae and microalgae which can be included in the feedstock stream can also be included within the biomass in the aqueous salt solution.

The expression “natural products” refers to products which are naturally produced or found within an environment or a living organism. Natural products can include those which are hydrophobic, hydrophilic or amphipathic.

In exemplary embodiments, the natural products are those which are naturally produced by a plant, a microbe, an algae or microalgae species which can be included within the feedstock stream or biomass. These natural products can include lipids, algal lipids, carotenoids, fatty acids, algal fatty acids, triacylglycerols, diacylglycerols, monoacylglycerols, oils, algal oils, chlorophyll, glycerol, phospholipids, carbohydrates, fibers, and proteins.

The expression “aqueous salt solution” refers to a solution containing water and at least one salt. The salt can be any one or combination of salts found in sea water, terminal lakes, or aquafers. In exemplary embodiments the aqueous salt solution is or includes: culture medium of the biomass.

The aqueous salt solution can include combinations of ions found in seawater.

The aqueous salt solution can contain concentrations of salts which range from trace amounts to saturating amounts. Suitable terms to describe the salinity or salt concentration of the aqueous salt solution range from fresh water, brackish water, salt water, brine, and saturated brine, respectively, as the salt concentration in the aqueous salt solution increase. The desired concentration of salt in the algal growth medium will depend on the type of feedstock present in the feedstock stream.

The expression “salinity” refers to the total amount of dissolved salts in the aqueous solution. Salts which can be dissolved and found in the aqueous solution include, but are not limited to, those found in natural waters such as sodium chloride, magnesium chloride, calcium and magnesium sulfates, bicarbonates, and carbonates. It is a standard practice to express salinity as parts per thousand (% 0), which is not a true percent but an approximation of the milligrams of salt per gram of water. In more general terms, salinity is indicated by the water source, such as a freshwater, a brackish water, a saline water, and a brine. Ranges of salinity are associated with these general terms and these ranges are defined as <0.05 wt % for freshwater, 0.05-3 wt % for brackish water, 3-5 wt % for saline water, and >5 wt % for a brine.

Various combinations of ions found in seawater can be included in the aqueous salt solution. Suitable ion combinations can be derived from one or more of the following sources including: water derived from streams, lakes, rivers, or other sources associated with fresh water; water derived from underground aquifers that can include various ion concentrations; water derived from industrial, agricultural, or municipal sources that can or cannot have received treatment; or water derived from brackish sources where fresh water is combined with sea water or ocean water in various proportions; sea water or ocean water that can be derived from the various seas and oceans located around the globe; water derived from terminal lakes; or combinations thereof. The combination of ions for the aqueous salt solution can be derived directly from these sources, or can be derived by evaporating the desired amount of water from any of these sources to leave the desired ion-rich solution for use as the aqueous salt solution. An example of an ion combination source is disclosed in U.S. Pat. No. 6,986,323, the contents of which are included herein by reference. Other examples include the evaporation of ancient sea waters that form terminal lakes, such as the Great Salt Lake in Utah, and that form various aquifers. The combination of ions can result up to and include crystallizers wherein sodium chloride ions are precipitated.

The aqueous salt solution can have a salinity that is about 5 wt % or greater than 5 wt %, about 6 wt % or greater than 6 wt %, about 7 wt % or greater than 7 wt %, for example at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, or at least about 25 wt %. In exemplary embodiments the aqueous salt solution is saturated with salt. In other exemplary embodiments, the aqueous salt solution can have a salinity that is about 5 wt % to about saturation, from about 10 wt % to saturation, from about 20 wt % to saturation, from about 5 wt % to about 20 wt %, from about 10 wt % to about 20 wt %, from about 5 wt % to about 15 wt %, from about 10 wt % to about 15 wt %, or from about 5 wt % to about 10 wt %. In an exemplary embodiment, the aqueous salt solution has a salinity greater than 5 wt %, greater than 10 wt %, greater than 15 wt % or greater than 20 wt %.

As used herein, wt % refers to a dry mass of a component in a solution in grams divided by 100 grams of the solution.

Salt may either be present in the aqueous salt solution or additional salt can be added to this stream to increase its salinity. The presence of salt, and specifically elevated salt compositions provides several advantages. First, the presence of salt in the aqueous biomass feed can reduce the solvent solubility in the raffinate layer, and thus reduce either potential solvent loss or solvent recovery costs. Second, the presence of salt in the aqueous biomass feed can increase the density of the raffinate layer, thus enhancing phase separation rates that leads to reduced decanter sizes. Third, the presence of salt in the aqueous salt solution can retard spoilage of the biomass in the processing step. All of these advantages, alone or in combination adds significant value to the extraction process.

The biomass in the aqueous salt solution can include or be a plant biomass, a microbial biomass, an algal biomass or any combination thereof.

All of the possible plants and/or microbes which can be included in the feedstock stream can also be included within the biomass in the aqueous salt solution.

All of the possible plant or microbial biomass which can be included in the feedstock stream include any plant or microbial biomass.

The biomass can also include or contain some or all of the natural products within the feedstock stream.

The biomass content in the feedstock stream can be as low as about 0.05 wt %, but it is preferred to be greater than about 0.5 wt % and even more preferred to be greater than 1 wt %. The maximum biomass content in the feedstock stream is limited as the maximum amount of biomass that allows the feedstock stream to flow, and this is less than about 20 wt %, or less than about 10 wt %.

The feedstock stream can contain a water content before the liquid-liquid-solid extraction process. The water content can be about 0.1 wt % to about 5 wt %, about 5 wt % to about 10 wt %, about 10 wt % to about 15 wt %, about 15 wt % to about 20 wt %, about 20 wt % to about 30 wt %, about 30 wt % to about 40 wt %, about 40 wt % to about 50 wt %, or about any range within 0.1 wt % to 50 wt % of the total weight of the feedstock stream. In exemplary embodiments, the feedstock stream can contain a water content before the liquid-liquid-solid extraction process that is greater than 50 wt % or about any range within 50 wt % to 99 wt %.

The biomass can include or be a conditioned biomass. As used herein “a conditioned biomass” refers to a biomass that has been treated with one or more conditioning processes before the liquid-liquid-solid extraction of the present disclosure. The suitable conditioning processes can include, but are not limited to, fracking, adsorptive bubble separation, filtration, deep bed filtration, belt pressing, screw pressing, centrifugation, adsorption, sedimentation, mechanical floatation, froth flotation, flocculation and combinations thereof.

The liquid-liquid-solid extraction process further includes a step of forming a dispersion by contacting (e.g., by intimately contacting) the feedstock stream with an extraction solvent in an extraction zone.

The term “dispersion” relates to a heterogeneous or homogeneous mixture containing the aqueous salt solution, the biomass, and the extraction solvent.

The extraction solvent must form a second liquid phase or layer with the aqueous salt solution in the separation zone. Suitable extraction solvents include, but are not limited to a non-polar solvent, a non-polar organic solvent, a dense gas solvent, an aqueous two-phase solvent, a deep eutectic solvent (DES) and/or a natural deep eutectic solvent (NADES) (such as choline chloride, glucose, lactic acid, malic acid, and/or any combination thereof), an ionic liquid, or any combination thereof or any combination of solvents such as miscible solvents. In an exemplary embodiment, the extraction solvent is or includes at least one or more of a non-polar solvent, a non-polar organic solvent, a dense gas solvent, an aqueous two-phase solvent, a deep eutectic solvent (DES), a natural deep eutectic solvent (NADES), an ionic liquid, or any combination thereof. In another exemplary embodiment, the extraction solvent is or includes at least one or more of a non-polar solvent, a non-polar organic solvent, a dense gas solvent, an aqueous two-phase solvent, a deep eutectic solvent (DES), a natural deep eutectic solvent (NADES), or any combination thereof. In another exemplary embodiment, the extraction solvent is or includes at least one or more of a non-polar solvent, a non-polar organic solvent, a dense gas solvent, an aqueous two-phase solvent, or any combination thereof. In another exemplary embodiment, the extraction solvent is or includes at least one or more of a non-polar solvent, a non-polar organic solvent, a dense gas solvent, or any combination thereof.

The extraction solvent is chosen such that its polarity is appropriate to extract the desired natural products. Thus, the optimal extraction solvent for the liquid-liquid-solid extraction process can depend on which natural products are desired to be extracted.

Any solvent system that forms a second immiscible liquid phase or layer with the feedstock stream can be used as the extraction solvent. These solvent systems should not adversely impact the quality or quantity of the natural products. These solvent systems can include synthetic and/or natural flavorants, edible oils, petrochemicals, dense gases, and combinations of these so long as the mixture of the solvent system and the biomass forms two immiscible liquid phases at a desired separation zone temperature. Some of these solvents are more desirable than others for various reasons as discussed below and the results obtained are not necessarily equivalent.

The solvent system can include petrochemical solvents due to their low viscosity and favorable solute molecular diffusivity. Natural oils are soluble in petrochemical solvents and concentrated extracts are possible. Suitable petrochemical solvents can include those that are disclosed in “Organic Solvents: Physical Properties and Methods of Purification”, edited by J. A. Riddick et al, Volume 2, Fourth Edition, ISBN Number 0-471-08467-0, such as 2-methyl oxolane. In exemplary embodiments, the petrochemical solvents can include, but are not limited to, aliphatic hydrocarbons (such as pentane, hexane, heptane, octane, nonane, decane, dodecane, cyclohexane, petroleum ether, their isomers, and mixtures thereof), aromatic hydrocarbons (including but not limited to benzene, toluene, xylene), alcohols (including, but not limited to butanol, pentanol, hexanol, octanol, dodecanol, cyclohexanol, benzyl alcohol, their isomers, and combinations thereof), ketones (including, but not limited to methyl isobutyl ketone, hexanone, heptanone, octanone, their isomers, and combinations thereof), esters (including, but not limited to methyl acetate, ethyl acetate, propyl acetate, butyl acetate, pentyl acetate, hexyl acetate, ethyl propionate, ethyl butyrate, ethyl valerate, their isomers, and combinations thereof). Combinations of petrochemical solvents can also be used if desired.

The petrochemical solvents can also contain one or more co-solvents to improve extractability of solutes. Examples of these co-solvents include methanol, ethanol, 1-propanol, 2-propanol, 1-hexanol, 2-methoxy ethanol, acetone, tetrahydrofuran, 1,4-dioxane, acetonitrile, dichloromethane, chloroform, dimethyl sulfoxide, formic acid, carbon disulfide, methylene chloride, amines, chelating agents, phase transfer catalysts and combinations thereof. The co-solvents can also be added to the feedstock stream to enhance recovery of a solute or hydrophobic natural product in the extraction solvent.

The edible oils which can be included within the solvent system can be chosen from those obtained from plant or animal sources, such as fish oils. Edible vegetable oil solvents include, but are not limited to, those derived from corn, olive, algae, soybean, flax, safflower, sunflower, palm, jatropha, coconut, other oils known in the art, and combinations thereof. Compared to petrochemical solvents, edible oils can be more viscous, and the solute molecular diffusivity is lower.

The solvent system can also include synthetic and natural flavorants. These flavorants can be more desirable than petrochemical solvents and edible oils if the natural products are to be used for human or animal consumption. Naturally derived flavorants have appeal in nutritional supplements. Flavorants classified by the Flavor and Extract Manufacturers Association, or FEMA, as Generally Recognized As Safe, or GRAS, do not have the drawbacks of petrochemical solvents in association with nutritional supplements. The presence of residual flavorant solvents in nutritional supplements is generally acceptable in comparison with petrochemical solvents, which reduces downstream purification and recovery costs. The flavorants can be chosen from those with have boiling points, viscosities, and molecular diffusivity properties comparable to petrochemical solvents. Examples of such flavorants include, but are not limited to, methyl-, ethyl-, propyl-, butyl-, isobutyl-, benzyl-, and octyl-esters with the carboxylic acid component of the ester including acetate, ethanoate, propionate, butyrate, hexanoate, caproate, heptanoate, octanoate, decanoate, cinnamate, and isovalerate. Other examples of flavorants which can be used include, but are not limited to, benzaldehyde, other aldehydes, limonene, and other terpenes. Combinations of flavorants can also be used, if desired.

Suitable dense gases which can be used as the extraction solvent include, but are not limited to, carbon dioxide, ethane, propane, butane, chlorofluorocarbons, and mixtures thereof. The dense gas extraction can be operated in any manner known in the art including leaching, batch extraction, and continuous countercurrent extraction as described in U.S. Pat. Nos. 6,106,720 and 5,932,101, the contents of which are incorporated herein by reference in their entirety. Additional suitable dense gases can be methane, isobutane, dimethyl ether, sulfur hexafluoride, ammonia, fluorocarbons, and mixtures thereof. Any combination of the above dense gases can also be used, if desired.

The dense gases can also contain one or more co-solvents to improve extractability of solutes. Examples of these co-solvents include methanol, ethanol, 1-propanol, 2-propanol, 1-hexanol, 2-methoxy ethanol, acetone, tetrahydrofuran, 1,4-dioxane, acetonitrile, dichloromethane, chloroform, dimethyl sulfoxide, formic acid, carbon disulfide, methylene chloride, amines, chelating agents, phase transfer catalysts and combinations thereof. Other examples of dense gases and co-solvents are listed in U.S. Pat. Nos. 4,345,976 and 5,490,884, the contents of which are incorporated herein by reference in their entirety. The co-solvents can also be added to the feedstock stream to enhance recovery of a solute or hydrophobic natural product in the extraction solvent.

The solvent system can also include an ionic liquid. Suitable ionic liquids include, but are not limited to, solvent systems that are in the liquid phase at the extraction temperature, those that include a cation and an anion, and those that are immiscible with a water-rich algal concentrate phase.

In exemplary embodiments, the feedstock stream or the biomass to the solvent ratio (e.g., a volumetric ratio) is from about 8 to about 0.1, from about 5 to about 0.2, such as 1 (e.g., a 1 to 1 volumetric ratio).

The feedstock stream can contact the extraction solvent for about 1 minute to 5 hours, or for about 2 minutes to 5 hours, and the contact time can differ based on the type of contactor used in the extraction zone. When centrifugal extractor is used for the extraction zone, the contact time can range from about 0.5 to 10 minutes, or less than 2 minutes. When an agitated vessel is used for the extraction zone, the contact time can range from about 1 minute to 5 hours, or between 2 and 120 minutes. Preferably, the contact time will be 5 to 60 minutes. The feedstock stream can contact the extraction solvent for about 2 to 180 minutes, about 5 to 180 minutes or about 10 to 60 minutes in a counter-current extraction column. In exemplary embodiments the dispersion is retained in the countercurrent extraction column for a residence time of about 2 minutes to about 2 hours.

The extraction zone can include a mixer, a static mixer, a settler, a co-current extraction column, a countercurrent extraction column, a centrifugal extractor, an emulsion phase contactor, or any combination thereof known in the art.

Suitable mixers for the extraction zone include agitated vessels where a mechanical agitator is used to intimately contact the feedstock stream and the extraction solvent. The mechanical agitator can include one or more impellers on a rotating shaft. Suitable impellers include, but are not limited to Rushton Turbines, flat-blade turbines, pitch-blade turbines, marine propellers, hydrofoils, impellers that are sold by Chemineer (Dayton Ohio), or SPX/Lightnin (Rochester, New York). Regardless of the type of impeller used, the degree of agitation required is important to efficient mass transfer of the solute. The degree of agitation required can be calculated by the minimum impeller speed to completely disperse one immiscible liquid in another, as defined by Skelland and Ramsay [1987 I&EC Res. 26, 1, 77-81], Skelland and Moeti [1989, I&EC Res. 28, 1, 122-127] and Skelland and Kanel [1993, I&EC Res. 29, 7, 1300-1306]. Static mixers of any design can also be used as the extraction zone. Suitable static mixers include, but are not limited to those produced by Chemineer in their Kenics line.

The extraction zone can be followed by a separation zone, and mass transfer can continue to occur while the dispersion is separating in the separation zone. In an exemplary embodiment, there is a balance between the amount of shear energy added in the extraction zone to generate an acceptable drop size distribution in the liquid-liquid dispersion in order to achieve acceptable mass transfer kinetics in the extraction zone, and reasonable decantation kinetics in the separation zone. Since the separation zone can be larger in volume than the extraction zone, it is advantageous not to add too much shear that generates a small drop size distribution that would increase the size of the decanter.

Suitable extraction columns which can be used as the extraction zone include, but are not limited to, those that are mechanically agitated and those that have stationary internals. The latter is preferred when the extraction solvent is a dense gas and/or the operating pressure of the extractor is elevated so that more expensive mechanical seals are needed. Suitable extraction columns with stationary internals can include, but are not limited to, packed, perforated plate, baffle tray, and combinations thereof. Suitable packings include structured or random packings that are known to those skilled in the art. Suitable mechanically agitated extraction columns can include, but are not limited to, the Karr reciprocating plate column, the York Scheibel column, and the rotating disc column, all made by Koch Modular Process Technology Corporation, which is located in Parasippany, N.Y., the Kuhni column, which is sold by Sulzer in Switzerland, the asymmetric rotating disc column, pulsed columns, and combinations thereof.

In exemplary embodiments wherein the extraction zone and separation zone are combined in a countercurrent extraction column, the raffinate layer will exit the column at one end while the solvent extract layer will exit the column at the opposite end. The rag layer can be removed from the countercurrent extraction column with either the raffinate layer or the solvent extract layer, or alternatively removed from the extraction column as a sidedraw. Any of the types of extraction columns described above can be used for countercurrent extraction. The feedstock stream can contact the extraction solvent for about 5 to 180 minutes, about 5 to 180 minutes or about 10 to 60 minutes in a counter-current extraction column.

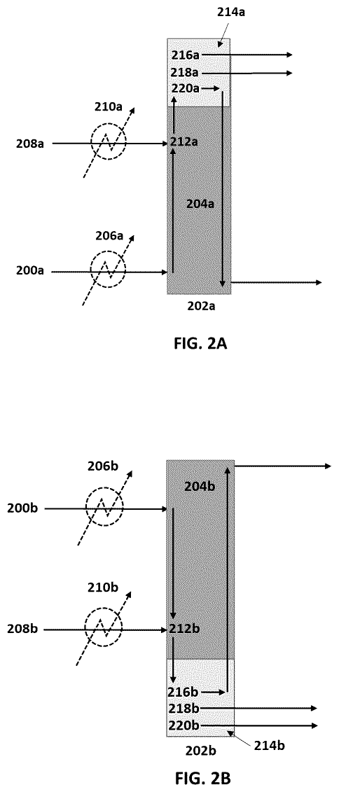

FIG. 2A depicts an exemplary embodiment where the extraction zone and separation zone are combined in a countercurrent extraction column, wherein the rag layer in removed with the solvent extract layer. The extraction solvent (200a) enters the countercurrent extraction column (202a) near the bottom of the extraction zone (204a). While entering the countercurrent extraction column (202a), the extraction solvent (200a) can undergo a heat exchange step (206a). The extraction solvent (200a) then travels up the extraction zone (204a).

While traveling up the extraction zone (204a), the extraction solvent (200a) intimately contacts the feedstock stream (208a) which enters the countercurrent extraction column (202a) at a point near the top of the extraction zone (204a). Before entering the extraction zone (204a), the feedstock stream (208a) can undergo a heat exchange step (210a). The interaction between the extraction solvent (200a) and feedstock stream (208a) produces a dispersion (212a), which travels further up the countercurrent extraction column (202a) towards the separation zone (214a).

Once in the separation zone (214a), the dispersion (212a) separates out into a solvent extract layer (216a), a rag layer (218a) and a raffinate layer (220a). The solvent extract layer (216a) and the rag layer (218a) are isolated and removed from the countercurrent extraction column (202a) while still in the separation zone (214a). In exemplary embodiments, the solvent extract layer (216a) and the rag layer (218a) are removed from the countercurrent extraction column (202a) as one stream instead of being separated and removed as two separate streams. The raffinate layer (220a) travels down the countercurrent extraction column (202a) back into the extraction zone (204a) where it is removed from the countercurrent extraction column (202a).

FIG. 2B depicts an exemplary embodiment where the extraction zone and separation zone are combined in a countercurrent extraction column, wherein the rag layer is removed with the raffinate layer. The feedstock stream (200b) enters the countercurrent extraction column (202b) near the top of the extraction zone (204b). While entering the countercurrent extraction column (202b), the feedstock stream (200b) can undergo a heat exchange step (206b). The feedstock stream (200b) then travels down the extraction zone (204b).

While traveling down the extraction zone (204b), the feedstock stream (200b) is intimately contacted with the extraction solvent (208b) in the extraction zone (204b). Before entering the extraction zone (204b), the extraction solvent (208b) can undergo a heat exchange step (210b). The interaction between the feedstock stream (200b) and the extraction solvent (208b) produces a dispersion (212b), which travels further down the countercurrent extraction column (202b) towards the separation zone (214b).

Once in the separation zone (214b), the dispersion (212b) separates out into a solvent extract layer (216b), a rag layer (218b) and a raffinate layer (220b). The raffinate layer (220b) and the rag layer (218b) are isolated and removed from the countercurrent extraction column (202b) while still in the separation zone (214b). In exemplary embodiments, the rag layer (218b) and the raffinate layer (220b) are removed from the countercurrent extraction column (202b) as one stream instead of being separated and removed as two separate streams. The solvent extract layer (216b) travels up the countercurrent extraction column (202b) back into the extraction zone (204b) where it is removed from the countercurrent extraction column (202b).

Suitable centrifugal extractors that can be used to provide both the extraction zone and the separation zone include, but are not limited to those produced by CINC, Alfal Lavel, Podbielniak, Robatel, Westfalia, and combinations of these centrifugal extractors.

Suitable emulsion phase contactors that can be used to provide both the extraction zone and the separation zone include, but are not limited to those produced by Schlumberger termed the NATCO dual frequency electrostatic treater.

The liquid-liquid-solid extraction process further includes passing the dispersion to a separation zone and separating the dispersion into multiple layers, the layers including a solvent extract layer, a raffinate layer and a rag layer. In exemplary embodiments, the rag layer is formed between the solvent extract layer and the raffinate layer.

The separation of the dispersion into multiple layers can be carried out or performed under a gravitational field or by decanting.

Separating the dispersion into multiple layers can occur in as little as 10 minutes to about 24 hours, at least 20 minutes to 12 hours, at least 30 minutes to 6 hours, or 40 minutes to 3 hours.

Separating the dispersion into multiple layers can occur or can be performed at a pressure ranging from atmospheric to supercritical conditions for the extraction solvent.

The separation zone can include or be a decanter which is configured to perform at least one or more of gravity settling, centrifugal settling, and/or combinations thereof to separate the dispersion into the multiple layers. In exemplary embodiments, the separation zone can include one or more fixed or moving separation aids like mesh pad coalescers, wire pad coalescers, structured packing, inclined plates, perforated plates, baffles, ultrasonic waves, acoustic waves, and/or combinations thereof.

In an exemplary embodiment the extraction zone includes a mixer and/or a countercurrent extraction column, and/or the separation zone includes a decanter.

The solvent extract layer can include at least one hydrophobic natural product present within the dispersion. These hydrophobic natural products can include one or more selected from the group including lipids, algal lipids, carotenoids, fatty acids, algal fatty acids, triacylglycerols, diacylglycerols, monoacylglycerols, oils, algal oils and combinations thereof.

The carotenoids can include beta-carotene, alpha-carotene, lutein, zeaxanthin, beta-cryptoxanthin, astaxanthin, phytoene, phytofluene, lycopene, and/or combinations thereof.

The solvent extract layer includes extraction solvent, algal oil, carotenoids, trace amounts of water and salt. The solvent extract layer can contain extraction solvent in amounts of more than 50 wt %, such as above 60 wt % or above 70 wt % of its total weight; algal oil in amounts than less 30 wt %, such as less than 20 wt % or less than 10 wt % of its total weight; carotenoids in amounts less than 5 wt %, such as less than 3 wt % or less than 1 wt % of its total weight; water in amounts less than 10 wt %, such as less than 5 wt % or less than 2 wt % of its total weight; and/or salt in amounts less than 3 wt %, such as less than 2 wt % or less than 1 wt % of its total weight.

The solvent extract layer can include limited amounts of lipid-depleted biomass and the aqueous salt solution.

The raffinate layer can include an aqueous salt solution depleted of hydrophobic natural products. The raffinate layer can possess a salt concentration of above 5 wt %, above 7 wt %, above 10 wt % above 15 wt %, above 18 wt % up to saturation. In an exemplary embodiment, the raffinate layer has a salinity of about 5 wt % or greater, about 10 wt % or greater, about 15 wt % or greater, or about 20 wt % or greater. In exemplary embodiments a salinity of the raffinate layer is about 5 wt % or greater than 5 wt %, about 6 wt % or greater than 6 wt %, about 7 wt % or greater than 7 wt %, for example at least about 8 wt %, at least about 9 wt %, at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, or at least about 25 wt %. In other exemplary embodiments the raffinate layer is saturated with salt. In more exemplary embodiments, the raffinate layer can have a salinity that is about 5 wt % to about saturation, from about 10 wt % to saturation, from about 20 wt % to saturation, from about 5 wt % to about 20 wt %, from about 10 wt % to about 20 wt %, from about 5 wt % to about 15 wt %, from about 10 wt % to about 15 wt %, or from about 5 wt % to about 10 wt %.

The raffinate layer can contain algal growth medium that is depleted in algal biomass and/or algal oils.

The raffinate layer can include water, salts, trace amounts of extraction solvent, trace amounts of algal biomass (such as less than 5 wt % or less than 1 wt % of its total weight) and/or trace amounts of algal oil (such as less than 5 wt % or less than 1 wt % of its total weight). The raffinate layer can contain water above 50 wt % (such as from 60 wt % to 90 wt %, 70 wt % to 85 wt % or 80 wt % to 90 wt %) or at about 50 wt % of its total weight, and/or the raffinate layer can contain salt from about 0.1 wt % to about 50 wt %, about 5 wt % to about 40 wt %, about 10 wt % to 30 wt %, and/or about 15 wt % to 26 wt % of its total weight. The raffinate can also contain trace amounts of the extraction solvent in both soluble and/or insoluble forms. The solubility of the extraction solvent in the raffinate layer influences the amount, or concentration, of the extraction solvent in the raffinate layer. The soluble amount of extraction solvent can be experimentally determined by measuring the solubility of the extraction solvent in the raffinate layer as a function of temperature, pressure, pH, salinity, and/or other factors known to those skilled in the art. The insoluble amount of the extraction solvent in the raffinate layer can be determined by the amount of entrainment of both extraction solvent droplets and the amount of extraction solvent that is associated with entrained algal biomass in the raffinate layer. Trace amounts of algal oil in the raffinate layer can be present in the solvent of the raffinate layer and/or in the algal biomass as unextracted oil.

The rag layer can form at any location between, above or below the solvent extract and raffinate layers, but can form between the solvent extract layer and the raffinate layer. It can be more difficult to separate the rag layer from the raffinate layer and the solvent extract layer if it forms between these two layers. The rag layer can include a lipid-depleted biomass. The lipid-depleted biomass can include at least one or more of, chlorophyll, glycerol, phospholipids, proteins, carbohydrates, fibers, and limited amounts of lipids, carotenoids and/or raffinate relative to the dispersion or any combination thereof. In exemplary embodiments, the rag layer contains at most about 45 wt % solvent extract layer, about 45 wt % raffinate layer and about 10 wt % lipid-depleted biomass. In an exemplary embodiment, the rag layer contains at most about 60 wt % solvent extract layer (such as from 30 wt % to 60 wt %, e.g., 40 wt % to 50 wt % or about 45 wt %), at most about 60 wt % raffinate layer (such as from 30 wt % to 60 wt %, e.g., 40 wt % to 50 wt % or about 45 wt %) and/or about from 2 wt % to 20 wt % lipid-depleted biomass (e.g., from 5 wt % to 10 wt %).

The rag layer can contain a majority of the algal biomass. Reducing the volume of the rag layer can be desirable to minimize the cost of further processing the rag layer to recover any entrained algal biomass.

The rag layer can include water, salt, extraction solvent, algae oil, and/or algal biomass. In exemplary embodiments, the rag layer is a mixture of three layers: 1) a layer containing algal biomass that has been depleted of non-polar compounds, 2) at least a portion of the solvent extract layer, and 3) at least a portion of the raffinate layer. The ratio of these three layers can vary depending on the conditions used to separate the dispersion into multiple layers. The layer containing the algal biomass that has been depleted of non-polar compounds can vary from about 0.01 wt % of the total weight of the rag layer to about 20 wt %. The solvent extract layer and the raffinate layer can vary from about 1 wt % to about 99 wt % of the total weight of the rag layer. In exemplary embodiments, the layer containing algal biomass that has been depleted of non-polar compounds is between about 1 and 10 wt % of the total weight of the rag layer, and the solvent extract layer and the raffinate layer range from about 10 wt % to 90 wt % of the total weight of the rag layer.

The rag layer can have an intermediate density between the solvent extract layer and the raffinate layer, so in decantation and/or centrifugation processes, it is located between the solvent extract layer and the raffinate layer.

In exemplary embodiments, the rag layer does not exist as a true thermodynamic phase and exists as a mixture of solid algal biomass, raffinate layer and solvent extract layer. The raffinate layer and the solvent extract layer can exist as true thermodynamic phases.

The liquid-liquid-solid extraction process can further include performing a heat exchange before the feedstock stream enters the extraction zone, before the extraction solvent enters the extraction zone, and/or before the dispersion enters the separation zone.

The liquid-liquid-solid extraction process includes isolating at least part of the solvent extract layer, at least part of the raffinate layer and/or at least part of the rag layer. The solvent extract layer can be e.g., overflowed or pumped out of the decanter, the rag layer can be pumped out of the decanter and/or the raffinate layer can be removed e.g., from the bottom of the separation zone or decanter.

The liquid-liquid-solid extraction process can include contacting the isolated solvent extract layer with an aqueous phase to remove any residual salt concentrations that can be present within the solvent extract layer.

The liquid-liquid-solid extraction process can include using a coalescer after formation of the dispersion.

The liquid-liquid-solid extraction process can include filtering the solvent extract layer after isolation to remove any entrained biomass, filtering the raffinate layer after isolation to remove any entrained biomass, and/or filtering the rag layer after isolation to remove any entrained solvent or any entrained aqueous salt solution.

The liquid-liquid-solid extraction process can include evaporating the extraction solvent from the solvent extract layer after isolation of the solvent extract layer.

The liquid-liquid-solid extraction process can include isolating the rag layer and can further include isolating the salt-laden lipid-depleted biomass from the rag layer.

The liquid-liquid-solid extraction process can further include removing any residual extraction solvent, salts and/or clay from the isolated salt-laden lipid-depleted biomass.

The liquid-liquid-solid extraction process can include pelletizing the isolated lipid-depleted biomass.

The liquid-liquid-solid extraction process can further include recycling the isolated lipid-depleted biomass into the feedstock stream.

The liquid-liquid-solid extraction process can include removing any entrained biomass, any entrained extraction solvent and/or any soluble solvent from the isolated raffinate layer.

The liquid-liquid-solid extraction process can be performed at a temperature of about 90° C. (e.g., 90±1-10° C.) or less, about 80° C. or less, about 70° C. or less, about 60° C. or less, or e.g., within a temperature range from 5 to 90° C., from 25 to 90° C., from 30 to 90° C., from 40 to 90° C., from 50 to 90° C., from 55 to 90° C., from 60 to 90° C., from 40 to 80° C., from 50 to 80° C., or from 60 to 80° C. It is preferable to operate the extraction at a temperature below 100° C. to preserve the algal oils and carotenoids.

In an exemplary embodiment, forming a dispersion by contacting the feedstock stream with an extraction solvent in an extraction zone and separating the dispersion into multiple layers in the separation zone can be performed at the same or different temperatures.

In an exemplary embodiment the separating of the dispersion into multiple layers occurs or is performed at a temperature of about 85° C. or less, about 80° C. or less, about 75° C. or less, about 70° C. or less, about 65° C. or less, about 60° C. or less, or within a temperature range from 35 to 90° C., from 40 to 90° C., from 45 to 90° C., from 50 to 90° C., from 55 to 90° C., from 60 to 90° C., from 30 to 80° C., from 35 to 80° C., from 40 to 80° C., from 45 to 80° C., from 50 to 80° C., from 55 to 80° C., or from 60 to 80° C., from 30 to 70° C., from 35 to 70° C., from 40 to 70° C., from 45 to 70° C., from 50 to 70° C., from 55 to 70° C., or from 60 to 70° C.

In an exemplary embodiment the separating of the dispersion into multiple layers occurs or is performed at a temperature of about 40° C. to 90° C., 50° C. to 90° C., 60° C. to 90° C., 35° C. to 80° C., 35° C. to 70° C. or 40° C. to 70° C.

In exemplary embodiments, the liquid-liquid-solid extraction process can be performed without addition of salt during the forming of the dispersion and during the separating of the dispersion into the multiple layers. For some exemplary embodiments, the extraction process includes performing the forming, passing, separating and isolating without an addition of salt. In other exemplary embodiments, the biomass or the feedstock stream containing the biomass in an aqueous salt solution does not include added salt.

The liquid-liquid-solid extraction process can be a continuous process. The liquid-liquid-solid extraction process can be configured as a continuous process wherein the forming, passing, separating and isolating steps are performed sequentially. Continuous operation can allow for the production of biofuels and/or other hydrophobic natural products with reduced capital and operating costs. In exemplary embodiments, the feedstock stream and the extraction solvent are intimately contacted so that the solvent receives the hydrophobic natural products. In other exemplary embodiments, the hydrophobic natural products are either pressed from the biomass or extracted with additional algal (or vegetable) oil. The resulting raffinate phase and the extract phases are separated so that the hydrophobic natural products can be further processed into desirable products.

A variety of extraction equipment components can be used for continuous extraction including: mixers and settlers, countercurrent extraction columns, centrifugal extractors, and other classes of extractors known in the art as described by Pratt et al., Selection, Design, PilotTesting, and Scale-Up of Extraction Equipment, Chapter 8, in Science and Practice of LiquidLiquid Extraction, Volume 1, Clarendon Press, Oxford, 1992, the contents of which are incorporated herein by reference. The feedstock stream and the extraction solvent can be contacted in a countercurrent or co-current flow.

Suitable centrifugal extractors can include, but are not limited to, those manufactured by GEA Westfalia Separator GmbH, which is headquartered in Oelde, Germany; Alfa Laval, with a location in Richmond, Virginia; Robatel, which is located in Pittsfield, Massachusetts; and Podbelniak, which is manufactured by Baker Perkins of Saginaw, Michigan.

Suitable other extraction equipment includes, but is not limited to hollow fiber membrane extractors and other novel extractor designs known in the art. In some cases, hollow fiber membrane extractors are used since they obviate the need to separate the solvent from the algal biomass.

Gravity settling is useful in a continuous extraction process. Separation of the multiple layers can be achieved in a centrifugal or gravitational force field, but gravity settling is usually of lower cost. A coalescer can be added to assist in the decantation. The raffinate can be further coalesced to recover any additional extraction solvent that can be entrained before being recycled to a bioreactor or returned to a pond, depending on the type of aquaculture practiced. A coalescer, liquid/liquid/solid centrifuge, flotation cell, and liquid/liquid cyclone can also be used to recover solvent from the aqueous salt solution, or the aqueous salt solution can be recycled to a flotation device for cleanup.

Suitable materials for the construction of the mixer, decanter, and/or extraction equipment include, but are not limited to, non-ferrous material, plastics, fiberglass, fiberglass reinforced plastic such as fiberglass reinforced HDPE, and combinations thereof. Non-ferrous materials are advantageous due to the salt content of the feedstock stream and the raffinate layer in the extraction process. The salinity of these components could cause stress corrosion cracking in ferrous materials, greatly increasing the maintenance required on the mixer, decanter, and extraction equipment. Plastic and fiberglass equipment is resistant to the effects of the elevated salinity and can be less expensive than equipment constructed of ferrous material.

The solvent extract layer, the lipid-depleted biomass, the rag layer, the raffinate layer or a combination thereof can be stabilized against degradation by any means known in the art including, but not limited to, one or more of the following methods: the addition of antioxidants, storage of the material in the absence of light exposure, storage under an inert environment such as nitrogen, argon, or carbon dioxide, and subjecting the material to a thermal cycle to destroy bacteria. Suitable antioxidants include, but are not limited to carotenoids, tertiary butyl hydroquinone (TBHQ), butylated hydroxyanisole (BHA), butylated hydroxytoluene (BHT), vitamin E, vitamin C, rosemary extracts, and combinations thereof.

Exemplary advantages of the liquid-liquid-solid extraction process include, but are not limited to, not drying the biomass prior to the hydrophobic natural products being extracted, traditional liquid-liquid extraction equipment can be used instead of expensive leaching equipment, salt does not need to be removed prior to the extraction process, and/or washing of the solvent extract layer can be accomplished in traditional liquid-liquid extraction equipment.

Any of a variety of products can be made from the biomass or lipid-depleted biomass including, but not limited to, biofuels, nutraceuticals, cosmaceuticals, wastewater treatment processes, spa products, animal feeds, human food, soil builders, chemical intermediates, specialty lipids, solar salt, and combinations thereof.

Biofuels that can be produced from high temperature processing of the biomass or lipid-depleted biomass include, but are not limited to, biodiesel, green diesel, renewable diesel, methane, alcohols, and dried algal biomass. Algal biodiesel is produced via any transesterification process known in the art, including those which utilize two immiscible liquid phases, and those that utilize a solid acid catalyst. Green diesel can be produced by hydrogenation, cracking, or a combination thereof of the algal oil or any derivative thereof in order to produce hydrocarbons that can be used directly in the existing diesel distribution system. Methane and/or hydrogen can be produced from the biomass or lipid-depleted biomass by any anaerobic process known in the art. Fermentation of the biomass or lipid-depleted biomass by any process known in the art can be used to produce methanol, ethanol, butanol, n-butanol, i-butanol, other alcohols, and combinations thereof. The biomass or lipid-depleted biomass can be torrified for the production of a soil builder or for use in combination with coal for power or steam generation. The biomass or lipid-depleted biomass can be dried and then gasified or combusted either by itself or in combination with coal or biomass.

The biomass or lipid-depleted biomass can be extracted to recover the lipids that can be used as an animal feed ingredient, renewable plastics, renewable polymers, renewable chemicals, nutraceutical, cosmaceutical, soap or components of a soap or detergent composition, and cosmetic ingredients, including, but not limited to carotenoids, omega fatty acids, and other lipids. For the production of solar salt, the biomass can be removed from solar salt works in order to improve the salt quality. The quality of sodium chloride, sodium carbonate, and other salts can be improved by this method. Biomass or lipid-depleted biomass stabilized with the high temperature treatment process can also be used in animal nutrition, especially for shrimp and fish aquaculture diets. The biomass or lipid-depleted biomass can also be treated with the high temperature process to stabilize it against degradation during transportation. Alternatively, high temperature processing could be used to stabilize the biomass or lipid-depleted biomass prior to its storage for carbon sequestration purposes. The biomass can be used to derive valuable chemical intermediates such as fatty acids for the production of polyurethanes.

Suitable animal feeds include, but are not limited to, feeds for shrimp, fish, shellfish, brine shrimp, chickens, poultry, cows, ducks, dogs, pigs, sheep, goats, and combinations thereof. The animal feeds can require the stabilized biomass to be dried, but in some cases, for example for use in shrimp and fish aquaculture diets, complete drying cannot be necessary as long as stabilization from the is sufficient.

Suitable dietary supplements include, but are not limited to alpha carotene, betacarotene, lutein, zeaxanthin, cryptoxanthin, phytoene, phytofluene, and the various cis- and trans-isomers and the various alpha, beta, gamma, delta isomers of the various carotenoids, and combinations thereof.

Suitable methods of carbon storage include, but are not limited to, burying the biomass or lipid-depleted biomass, sinking it, torifying it and using it as a soil builder, or combinations thereof.

Suitable methods for water and wastewater treatment include, but are not limited to, removal of BOD (biological oxygen demand), and or TOC (total organic carbon) from a water stream. This can be useful for municipal wastewater treatment processes, and it can be important for the treatment of brines being used for the production of sodium chloride salt and other salts via evaporation.

Suitable methods to process the biomass or lipid-depleted biomass into useful compounds include, but are not limited to torification, gasification, liquefaction, fermentation, drying, combustion, burial, and combinations thereof. Suitable applications of the torified biomass includes, but is not limited to, a soil builder and a material to be combined with coal, wood, or other combustible material for power generation. Suitable applications of gasified biomass includes, but is not limited to, the production of the entire suite of products that can be produced via syngas chemistry, as described by the Gasification Technologies Council. Suitable products from syngas include, but are not limited to, chemicals, fertilizers, power generation, substitute natural gas, hydrogen, and transportation fuels. Suitable chemicals include, but are not limited to, hydrogen, carbon monoxide, methanol, dimethyl ether, acetic acid, propionic acid, butyric acid, acetic anhydride, methyl acetate, ethylene, propylene, olefins, and combinations thereof. Suitable fertilizers that can be produced from the syngas include, but are not limited to ammonia, ammonium nitrate, urea, and others known in the art. Suitable substitute natural gas can be generated from the syngas produced by gasifying algal biomass or lipid-depleted biomass, and this includes methane. Suitable liquid fuels include gasoline, diesel fuel, jet fuels, and combinations thereof. All of the chemicals that are produced by Eastman Chemicals and by Sasol via their gasification processes can also be produced by the gasification of biomass or lipid-depleted biomass. Products produced by the utilization of syngas can also be produced by gasification of biomass. Illustrative processes are described in U.S. Pat. No. 6,310,260, the contents of which are incorporated herein by reference, include, for example, hydroformylation, hydroacylation (intramolecular and intermolecular), hydrocyanation, hydroamidation, hydroesterification, aminolysis, alcoholysis, hydrocarbonylation, reductive hydroformylation, hydrogenation, olefin oligomerization, hydroxycarbonylation, carbonylation, olefin isomerization, transfer hydrogenation and the like. Other processes involve the reaction of organic compounds with carbon monoxide, or with carbon monoxide and a third reactant, e.g., hydrogen, or with hydrogen cyanide, in the presence of a catalytic amount of a metal-organophosphorus ligand complex catalyst. More advantageous processes include hydroformylation, hydrocyanation, hydrocarbonylation, hydroxycarbonylation and carbonylation.

### EXAMPLES

The present disclosure will be described in more detail with reference to the following Examples, which shows exemplary embodiments in accordance with the present disclosure. The present disclosure is not limited to these exemplary embodiments.

### Example 1

Algal concentrate was pumped with a FMI Lab Pump Model QSY from a 2 L agitated vessel into a 1 L round bottomed flask at an average rate of 24 ml/min. The vessel was agitated with an impeller attached to a variable speed motor. Heptane was pumped with an FMI Lab Pump Model QSY from a 1 L graduated cylinder into the same 1 L round bottomed flask at an average rate of 7 ml/min. The average algal concentrate to solvent ratio was 3.4 on a volumetric basis. The 1 L round bottomed flask was agitated with an impeller driven by a variable speed motor. The flask was jacketed, with the jacket containing water at 60° C. The mixture of algal concentrate and heptane was pumped out of the 1 L flask at a sufficient rate to keep the volume in the 1 L flask uniform. The mixture was pumped into a 3.5 L vertical decanter. The decanter was jacketed with the jacket containing water at 60° C. The mixture decanted into three phases, the top phase being the solvent extract layer, the middle phase being the rag layer, and the bottom phase the raffinate layer. The solvent extract layer overflowed out of the decanter, through a filter and into an Erlenmeyer flask. The rag layer was pumped out of the decanter with an FMI Lab Pump Model QSY into a second decanter. The raffinate layer was removed through a swing arm of variable height attached to the bottom of the decanter and discarded. The levels of the interfaces of the layers in the decanter were controlled by adjusting the speed of the rag layer pump and the height of the raffinate layer swing arm. The second decanter was a 4 L beaker heated by a hot plate. The same three layers in the first decanter were present in the second decanter. The interfaces of the layers in the second decanter were controlled by pumping out either the raffinate layer and discarding it, or the solvent extract layer to the filter and Erlenmeyer flask. Defatted algal biomass was collected out of the second decanter. The solvent extract layer was pumped to the heated flask of an evaporator with a FMI Lab Pump Model QSY. The evaporator included a 500 ml heated flask, a condenser, and a 1 L solvent collection flask. The heated flask was kept at a temperature of 60° C. with an electric heating mantle and system was kept at a pressure below 100 mmHg with a vacuum pump. The condenser was cooled with water. Heptane evaporated in the heated flask, condensed in the condenser and was recovered into the solvent collection flask. Heptane recovered in this process was recycled to the 1 L graduated cylinder. Algal oil recovered in the evaporation process was drained out of the heated flask through a stopcock in the bottom of the flask.

### Example 2

Algal concentrate with a salt concentration of about 22 wt % NaCl salt and heptane were intimately contacted in a 1 to 1 volumetric ratio at 22° C. and were charged to a vessel and allowed to decant by gravity at 22° C. The decantation data is tabulated in Table 1 below. The table gives information on the decantation time in minutes, the location of the top interface between the solvent extract layer and the rag layer in terms of dimensionless depth, and the location of the bottom interface between the rag layer and the raffinate layer in terms of dimensionless depth.

TABLE 1
22 C Decantation Data

| | Top | Bot |
|---|---|---|
| minutes | norm | norm |
| 0 | 1.00 | 0.00 |
| 1 | 0.98 | 0.01 |
| 2 | 0.98 | 0.02 |
| 5 | 0.94 | 0.09 |
| 10 | 0.94 | 0.14 |
| 22 | 0.91 | 0.31 |
| 43 | 0.90 | 0.33 |
| 161 | 0.75 | 0.38 |
| 649 | 0.52 | 0.40 |
| 650 | 0.51 | 0.39 |

### Example 3

Algal concentrate with a salt concentration of about 22 wt % NaCl salt and heptane were intimately contacted in a 1 to 1 volumetric ratio at 46° C. and were charged to a vessel and allowed to decant by gravity at 46° C. The decantation data is tabulated in Table 2 below. The table gives information on the decantation time in minutes, the location of the top interface between the solvent extract layer and the rag layer in terms of dimensionless depth, and the location of the bottom interface between the rag layer and the raffinate layer in terms of dimensionless depth.

TABLE 2
46 C Decantation Data

| | Top | Bot |
|---|---|---|
| minutes | norm | norm |
| 0 | 1.00 | 0.00 |
| 1 | 0.94 | 0.01 |
| 2 | 0.91 | 0.02 |
| 5 | 0.90 | 0.09 |
| 10 | 0.90 | 0.22 |
| 20 | 0.90 | 0.31 |
| 161 | 0.54 | 0.37 |
| 320 | 0.51 | 0.40 |
| 321 | 0.47 | 0.38 |

### Example 4

Algal concentrate with a salt concentration of about 22 wt % NaCl salt and heptane were intimately contacted in a 1 to 1 volumetric ratio at 60° C. and were charged to a vessel and allowed to decant by gravity at 60° C. The decantation data is tabulated in Table 3 below. The table gives information on the decantation time in minutes, the location of the top interface between the solvent extract layer and the rag layer in terms of dimensionless depth, and the location of the bottom interface between the rag layer and the raffinate layer in terms of dimensionless depth.

TABLE 3
60C Decantation Data

| | top | bot |
|---|---|---|
| minutes | norm | norm |
| 0 | 1.00 | 0.00 |
| 1 | 0.96 | 0.03 |
| 2 | 0.96 | 0.09 |
| 4 | 0.95 | 0.13 |
| 8 | 0.94 | 0.24 |
| 20 | 0.84 | 0.35 |
| 22 | 0.65 | 0.26 |
| 40 | 0.60 | 0.35 |
| 41 | 0.49 | 0.34 |
| 80 | 0.49 | 0,36 |
| 81 | 0.47 | 0.34 |
| 160 | 0.47 | 0.37 |
| 161 | 0.47 | 0.37 |

### Example 5: Effect of Temperature on Decantation

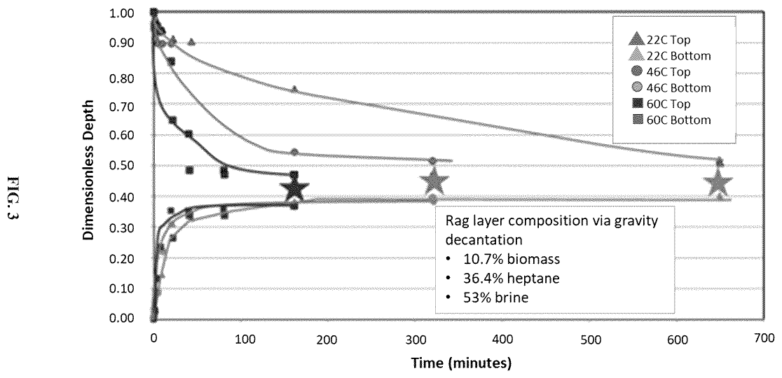

The decantation data collected from Examples 2, 3, and 4 are combined into a single graph shown in FIG. 3. The Y-axis is the dimensionless depth of the liquid in the vessel, where unity represents the gas-liquid interface and the origin marks the bottom of the vessel. The X-axis is the decantation time in minutes. The top three curves represent the interface level between the solvent extract layer and the rag layer as a function of temperature. The decantation time decreases as the temperature is increased from 22° C. to 46° C. and then to 60° C. The bottom three curves represent the measured location of the interface between the rag layer and the raffinate layer. Again, the decantation time decreases as the temperature increases from 22° C. to 46° C. and then to 60° C. The decantation time at 22° C., 46° C., and 60° C. were measure to be more than 650 minutes, more than 300 minutes, and about 150 minutes, respectively. Thus, elevating temperature significantly reduced the gravity decantation time.

### Examples 6-17

### Exemplary Procedure for the Extraction Step in Examples 6-17

The following experimental equipment and procedures were used for Examples 6-17. The liquid-liquid-solid extraction process was performed in a mixer-settler unit (a combined contacting and separating unit), for the isolation of natural products from algal biomass, via cell membrane disruption, in an aqueous brine solution with a solvent. The mixing and settling were performed in the same vessel with the following geometrical parameters: (1) liquid depth in vessel to vessel diameter ratio=1; (2) baffle width=0.1× vessel diameter, with four equally spaced baffles that extended from the gas-liquid interface to the start of the bottom dish; (3) impeller diameter=0.5× vessel diameter; (4) 3-blade HE-3 impeller from Chemineer (Dayton, Ohio) was centrally mounted in the vessel, (5) a variable speed drive was used to control and set the agitation rate, (6) a condenser was used to retain the solvent while allowing the system to be swept with an inert gas, (7) the jacketed agitated vessel was fitted with sight glasses so that the mixing and decantation steps could be visually observed, and (8) the vessel was cylindrical with a shallow dish at the bottom with a bottom drain valve that is exemplary of agitated vessels used commercially. This agitated vessel served as both the mixer and settler in these examples. Thus, the impeller was operated during the mixing step, and it was turned off to initiate the settling step. The algal feed included algal biomass and brine that was initially charged to the agitated vessel. The heat transfer fluid in the jacket on the agitated vessel was set to the desired temperature for the run, and agitation was started at a rate to facilitate proper heat transfer. When the algal feed has reached the desired temperature for the run, the agitation rate was then increased to the desired impeller speed for the experiment, and the flow patterns were established with the algal feed before the solvent was rapidly added to the agitated vessel at time zero for mixing. After the prescribed mixing time was reached, the impeller operation was stopped at time zero for decantation. The decantation was allowed to continue until the gravity decantation process approached 95% of the equilibrium interface levels.

### Mixer-Settler Extraction of Examples 6-16

A 1 wt % dispersion of Dunaliella salina biomass in aqueous brine including 20 wt % sodium chloride salt was charged to the agitated vessel as the algal feed. Agitation and heating commenced to allow the algal feed to reach 50° C. The impeller speed was increased to the desired set point, and the solvent (hexane) was rapidly added to achieve a solvent to feed volumetric ratio of 0.25. The impeller speed setpoint was 150% of the minimum impeller speed [Nmin] to completely disperse two immiscible liquid phases as defined by Skelland and Ramsay (Ind. Eng. Chem. Res. 1987, 26, 1, 77-81), the contents of which are hereby incorporated by reference in its entirety. The liquid-liquid-solid dispersion was agitated for 10 minutes to facilitate mass transfer, and then the agitation ceased and decantation commenced. The carotenoid yield was 15 wt % and total fatty acid yield 25%.

The extraction of Example 17 was performed using the above process but the Dunaliella salina biomass was grown at stressed conditions. The results of Examples 6-17 are depicted below in Table 4

TABLE 4
Extraction Data for Examples 6-17

| | | | D. | | | | H. | | |
|---|---|---|---|---|---|---|---|---|---|
| | B. | | Impeller | | F. | G. | Biomass | | J. |
| A. | Feed | C. | speed | | S:F | Mixing | loss in | I. | Total fatty |
| Ex. | [wt % | Temp. | [% of | E. | ratio | Time | raffinate | Carotenoid | acid yield |
| nr | solids] | [° C.] | Nmin] | Solvent | (vol) | [min] | [wt %] | yield (wt %) | (wt %) |
| 6 | 1 | 50 | 150 | Hexane | 0.25 | 10 | 13 | 15 | 25 |
| 7 | 1 | 50 | 150 | Hexane | 0.18 | 10 | 15 | 16 | 22 |
| 8 | 1 | 50 | 150 | Hexane | 0.11 | 10 | 17 | 14 | 13 |
| 9 | 1 | 50 | 150 | Hexane | 0.25 | 5 | 12 | 17 | 20 |
| 10 | 1 | 50 | 150 | Hexane | 0.25 | 20 | 11 | 22 | 38 |
| 11 | 3 | 50 | 150 | Hexane | 0.25 | 10 | 5 | 22 | 17 |
| 12 | 2 | 50 | 150 | Hexane | 0.25 | 10 | 7 | 23 | 20 |
| 13 | 2 | 70 | 150 | Heptane | 0.25 | 15 | 6 | 24 | 25 |
| 14 | 5 | 65 | 150 | Ethyl | 0.20 | 15 | 3 | 95 | 74 |
| | | | | acetate | | | | | |
| 15 | 7 | 75 | 150 | Limonene | 0.20 | 15 | 2 | 45 | 76 |
| 16 | 6 | 75 | 150 | Heptane | 0.20 | 15 | 6 | 32 | 31 |
| 17 | 2 | 75 | 150 | Heptane | 0.25 | 15 | 3 | 86 | 49 |

### Table 4 Column Notations

A. Example number
B. Weight-% biomass in the Feed
C. Extraction and Decantation Temperature in ° C.
D. Impeller speed as % of Nmin
E. Solvent
F. Solvent: Feed volumetric ratio
G. Agitation Time in minutes (min)
H. Approximate biomass loss in raffinate (wt %)
I. Carotenoid yield (wt %)
J. Fatty acids yield (wt %)

### Example 18

Algal biomass including 3 wt % Dunaliella salina in a 20 wt % salt in water solution was fed to the top of an active section of a Karr reciprocating plate extraction column operating in continuous counter-current mode. Heptane solvent was fed to the bottom of the active section of the extraction column. The solvent to feed volumetric ratio was 1.2, and the extraction temperature was held constant at 75° C. The ratio of the column diameter to active section length was 120. The carotenoid recovery in the extract was 77 wt % and the total fatty acid recovery in the extract was 54 wt %. The rag layer exited the extraction process as a side stream, and it included most of the algal biomass. The mean residence time in the active section of the extraction column was 10 minutes.

### Example 19

Algal biomass including 1.5 wt % Dunaliella salina in a 20 wt % salt in water solution was fed to the top of an active section of a Karr reciprocating plate extraction column operating in continuous counter-current mode. Heptane solvent was fed to the bottom of the active section of the extraction column. The solvent to feed volumetric ratio was 0.76, and the extraction temperature was held constant at 75° C. The ratio of the column diameter to active section length was 120. The carotenoid recovery in the extract was 91 wt % and the total fatty acid recovery in the extract was 62 wt %. The rag layer exited the extraction process as a side stream, and it included most of the algal biomass. The mean residence time in the active section of the extraction column was 10 minutes.

### Example 20

Algal biomass including 0.5 wt % Dunaliella viridis in a 20 wt % salt in water solution was fed to the top of the active section of a Scheibel extraction column operating in continuous co-current mode. Heptane solvent was fed to the bottom of the active section of the extraction column. The solvent to feed volumetric ratio was 1.2, and the extraction temperature was held constant at 70° C. The ratio of the column diameter to active section length was 40. After decantation, the carotenoid recovery in the extract was 21 wt % and the total fatty acid recovery in the extract was 53 wt %. The rag layer exited the extraction process as a side stream, and it included most of the algal biomass. The mean residence time in the active section of the extraction column was 15 minutes.

### Example 21

Algal biomass including 9 wt % fractured Haematococcus pluvialis cells in a 10 wt % salt in water solution was fed to the top of the active section of an extraction column packed with 316 stainless steel structured packing and operating in continuous countercurrent mode. A mixture of hexane and ethyl acetate (1:1 mass ratio) solvent was fed to the bottom of the active section of the extraction column. The solvent to feed volumetric ratio was 2.5, and the extraction temperature was held constant at 50° C. The ratio of the column diameter to active section length was 40. After decantation, the carotenoid recovery in the extract was 43 wt % and the total fatty acid recovery in the extract was 37 wt %. The rag layer exited the extraction process as a side stream, and it included most of the algal biomass. The mean residence time in the active section of the extraction column was 25 minutes.

### Example 22

Algal biomass including 5 wt % of pre-ruptured Blakeslea trispora in a 9 wt % salt in water solution was fed to the top of the active section of a Kuhni extraction column operating in continuous countercurrent mode. Heptane solvent was fed to the bottom of the active section of the extraction column. The solvent to feed volumetric ratio was 3, and the extraction temperature was held constant at 75° C. The ratio of the column diameter to active section length was 40. After decantation, the carotenoid recovery in the extract was 84 wt % and the total fatty acid recovery in the extract was 68 wt %. The rag layer exited the extraction process as a side stream, and it included most of the algal biomass. The mean residence time in the active section of the extraction column was 18 minutes.

### Example 23

Algal biomass including 1.5 wt % Dunaliella salina in a 20 wt % salt in water solution was fed to the top of the active section of a Karr reciprocating plate extraction column operating in continuous counter-current mode. Heptane solvent was fed to the bottom of the active section of the extraction column. The solvent to feed volumetric ratio was 0.76, and the extraction temperature was held constant at 50° C. The ratio of the column diameter to active section length was 120. The extract and rag layer were fed to an external decanter which was held at 75° C. where the resulting rag layer included a majority of the algal biomass. The carotenoid recovery in the extract was 91 wt % and the total fatty acid recovery in the extract was 51 wt %. The rag layer exited the extraction process as a side stream, and it included most of the algal biomass. The mean residence time in the active section of the extraction column was 10 minutes.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

1. A liquid-liquid-solid extraction process for isolating natural products from a feedstock stream containing a biomass in an aqueous salt solution, the process comprising:
forming a dispersion by contacting the feedstock stream with an extraction solvent in an extraction zone;
passing the dispersion to a separation zone;
separating the dispersion into multiple layers at a temperature of about 30° C. to about 90° C., the layers including: a solvent extract layer containing at least one hydrophobic natural product and the extraction solvent, a raffinate layer containing the aqueous salt solution, and a rag layer containing a lipid-depleted biomass; and
isolating at least part of the solvent extract layer, at least part of the raffinate layer and/or at least part of the rag layer.

2. The process of claim 1, wherein the biomass is or comprises:
at least one or more of a plant biomass, a microbial biomass, an algal biomass and/or any combination thereof.

3. The process of claim 1, wherein the extraction solvent is or comprises at least one or more of a non-polar solvent, a non-polar organic solvent, a dense gas solvent, an aqueous two-phase solvent, a deep eutectic solvent (DES), a natural deep eutectic solvent (NADES), an ionic liquid, or any combination thereof.

4. The process of claim 3, wherein the extraction solvent is or comprises at least one or more of a non-polar solvent, a non-polar organic solvent, a dense gas solvent, an aqueous two-phase solvent, or any combination thereof.

5. The process of claim 1, wherein the extraction zone is or comprises at least one or more of a mixer, a static mixer, a settler, a co-current extraction column, a countercurrent extraction column, a centrifugal extractor, an emulsion phase contactor, or any combination thereof.

6. The process of claim 1, wherein the extraction zone is a countercurrent extraction column, the process optionally comprising: retaining the dispersion in the countercurrent extraction column for a residence time of about 2 minutes to about 2 hours.

7. The process of claim 1, comprising:
performing the separating of the dispersion into multiple layers by gravity under a gravitational field.

8. The process of claim 1, comprising:
performing the separating of the dispersion into multiple layers by decanting.

9. The process of claim 1, wherein the separation zone is a decanter and wherein the separating optionally comprises: at least one or more of gravity settling, centrifugal settling, and/or combinations thereof to separate the dispersion into the multiple layers.

10. The process of claim 1, wherein the hydrophobic natural products are or comprise at least one of lipids, algal lipids, carotenoids, fatty acids, algal fatty acids, triacylglycerols, diacylglycerols, monoacylglycerols, oils, algal oils or any combination thereof.

11. The process of claim 1, wherein the aqueous salt solution has a salinity greater than 5 wt %, greater than 10 wt %, greater than 15 wt % or greater than 20 wt %.

12. The process of claim 1, wherein
the aqueous salt solution is or comprises:
culture medium of the biomass.

13. The process of claim 1, wherein
the extraction process comprises:
performing the forming, passing, separating and isolating without an addition of salt.

14. The process of claim 1, wherein the lipid-depleted biomass comprises at least one or more of chlorophyll, glycerol, phospholipids, carbohydrates, fibers, proteins or combinations thereof.

15. The process of claim 1, comprising:
performing the forming, passing, separating and isolating as a continuous process, wherein the forming, passing, separating and isolating are performed sequentially.

16. The process of claim 1, comprising:
isolating the solvent extract layer; and
contacting the isolated solvent extract layer with an aqueous phase to remove any residual salt concentrations in the solvent extract layer.

17. The process of claim 1, comprising:
using a coalescer after formation of the dispersion.

18. The process of claim 1, comprising:
filtering the solvent extract layer after isolation to remove any entrained biomass;
filtering the raffinate layer after isolation to remove any entrained biomass; and/or
filtering the rag layer after isolation to remove any entrained solvent or any entrained aqueous salt solution.

19. The process of claim 1, comprising:
evaporating the extraction solvent from the solvent extract layer after isolation of the solvent extract layer.

20. The process of claim 1, comprising:
allowing the feedstock stream to contact the extraction solvent for about 1 minute to about 5 hours.

21. The process of claim 1, comprising:
performing the separating for at least 10 minutes to about 24 hours.

22. The process of claim 1, comprising:
performing the separating at a pressure ranging from atmospheric to supercritical conditions for the extraction solvent.

23. The process of claim 1, comprising:
isolating the rag layer, and isolating the lipid-depleted biomass from the rag layer.

24. The process of claim 23, comprising:
removing any residual extraction solvent, salts and/or clay from the isolated lipid-depleted biomass.

25. The process of claim 23, comprising:
pelletizing the isolated lipid-depleted biomass.

26. The process of claim 1, comprising:
isolating the raffinate layer; and removing any entrained biomass, any entrained extraction solvent and/or any soluble solvent from the isolated raffinate layer.

27. The process of claim 1, wherein the feedstock steam contains a water content before extracting.

28. The process of claim 1, comprising:
performing a heat exchange before the feedstock stream enters the extraction zone, before the extraction solvent enters the extraction zone, and/or before the dispersion enters the separation zone.

29. The process of claim 1, wherein the biomass is or comprises conditioned biomass.

30. The process of claim 1, wherein the rag layer is formed between the solvent extract layer and the raffinate layer.

31. The process of claim 1, wherein the separating of the dispersion into multiple layers occurs in a temperature regulated environment.

32. The process of claim 1, wherein the raffinate layer has a salinity of about 5 wt % or greater, about 10 wt % or greater, about 15 wt % or greater, or about 20 wt % or greater.

33. The process of claim 1, wherein the rag layer contains at most about 60 wt % solvent extract layer (such as from 30 wt % to 60 wt %, e.g. 40 wt % to 50 wt % or about 45 wt %), at most about 60 wt % raffinate layer (such as from 30 wt % to 60 wt %, e.g. 40 wt % to 50 wt % or about 45 wt %) and/or about from 2 wt % to 20 wt % lipid-depleted biomass (e.g. from 5 wt % to 10 wt %).

34. The process of claim 1, wherein the raffinate layer contains salt from about 0.1 wt % to about 50 wt %, from about 5 wt % to about 40 wt %, from about 10 wt % to 30 wt %, or from about 15 wt % to 26 wt % of the total weight of the raffinate layer.

35. The process of claim 1, wherein the separating of the dispersion into multiple layers occurs at a temperature of about 40° C. to 90° C., 50° C. to 90° C., 60° C. to 90° C., 35° C. to 80° C., 35° C. to 70° C. or 40° C. to 70° C.

36. The process of claim 1, wherein the extraction zone comprises a mixer and/or a countercurrent extraction column, and/or the separation zone comprises a decanter.
